(19) United States
(12) Patent Application Publication
(10) Pub. No.: US 2018/0280340 A1
(43) Pub. Date: Oct. 4, 2018

(54) METHOD OF TREATING AND PREVENTING INFECTIONS

(71) Applicants: Konstantin Zubovskiy, Morristown, NJ (US); Michael Chikindas, Highland Park, NJ (US)

(72) Inventors: Konstantin Zubovskiy, Morristown, NJ (US); Michael Chikindas, Highland Park, NJ (US)

(21) Appl. No.: 15/940,662

(22) Filed: Mar. 29, 2018

Related U.S. Application Data
(60) Provisional application No. 62/479,261, filed on Mar. 30, 2017.

(51) Int. Cl. A61K 31/327; A61K 9/00; A61P 31/04; A61P 29/00; A61P 17/06; A61P 19/02; A61P 1/00

(52) U.S. Cl. CPC A61K 31/327; A61K 9/0014; A61P 31/04; A61P 1/00; A61P 17/06; A61P 19/02; A61P 29/00

(57) ABSTRACT

Described are methods and pharmaceutical compositions for treating or preventing bacterial infections and other disease and conditions caused by bacterial imbalance or mediated via human microbiota. For example, described are pharmaceutical compositions comprising sub-minimum inhibitory concentrations of reactive oxygen species sufficient for arresting virulence through inhibition of quorum sensing, thereby modifying microbiota to a healthy state

### CROSS-REFERENCE TO RELATED APPLICATIONS

This application claims the benefit of priority to U.S. Provisional Patent Application 62/479,261 filed 30 Mar. 2017, which is incorporated by reference herein in its entirety for all purposes.

### BACKGROUND

### 1. Field of the Discovery

Described are methods and pharmaceutical compositions for treating or preventing bacterial infections and other disease and conditions caused by bacterial imbalance or mediated via human microbiota. For example, described are pharmaceutical compositions comprising sub-minimum inhibitory concentration (sub-MIC); i.e., concentrations that are below established MIC, of reactive oxygen species (ROS) sufficient for arresting virulence through inhibition of quorum sensing, thereby modifying microbiota to a healthy state

### 2. Background Information

The emergence of multidrug-resistant pathogens and the prevalence of biofilm-related infections have generated a demand for alternative anti-microbial therapies. Bacterial infection remains a major challenge to healthcare and is responsible for significant morbidity and mortality. This situation is becoming complicated by an increasingly aging and susceptible population and large numbers of bacterial isolates, which have developed resistance to antibiotics. Bacteria that form biofilms and colonize or infect medical devices or wounds are particularly hard to treat as biofilms are inherently highly antibiotic resistant. Most recurrent infections of biological surfaces have a component where bacteria form a biofilm, and as a result, elimination of biofilm formation and prevention of biofilm-development are highly important.

Various microbes, from commensal to pathogenic, reside in the human body. Not only are they interacting with their host, but also these different microorganisms are interacting with each other. This interaction sometimes causes dysbiosis, which refers to microbial imbalance inside the body, i.e., increased levels of harmful bacteria and reduced levels of the beneficial bacteria (World J Gastroenterol. 2015; 21(40): 11450-11457. Dysbiotic infection in the stomach.).

These microbes coordinate their formation of biofilms and their expression of virulence factors through quorum sensing (QS), a system that regulates gene expression at high cell densities and that plays a key role in the establishment of bacterial infections. Microbial colonies found in human body are normally beneficial, but could be parasitic, commensal, or symbiotic. The beneficial bacterial colonies also protect the body from the penetration of pathogenic microbes by competing with pathogens for space and nutrition. The alteration of human microbiota and particularly changes in gut microbiota, have been demonstrated to interfere with immunity and immune responses and therefore playing a role in wide range of diseases involving systemic inflammation.

Interfering with the quorum-sensing systems has been proposed as an alternative to traditional antibiotics for the eradication of bacterial infections. Various efforts have been made in biofilm disruption by quorum sensing interference but current methods have limitations in the implementation of these novel therapeutic options (Curr Top Med Chem. 2017, Exploiting Quorum Sensing Inhibition for the Control of Pseudomonas Aeruginosa and Acinetobacter Baumannii Biofilms).

One of the examples of bacterial infections due to microbiota imbalance is Bacterial Vaginosis (BV). Vaginal infections are a common problem among women. BV is the most common genital tract infection in women during their reproductive years and it has been associated with serious health complications, such as preterm delivery and acquisition or transmission of several sexually transmitted agents. BV accounts for 45% of symptomatic cases and estimated to be present in 15% of asymptomatic sexually active women. BV is a polymicrobial vaginal infection characterized by a reduction of beneficial lactobacilli and a significant increase in number of anaerobic bacteria, including Gardnerella vaginalis, Atopobium vaginae, Mobiluncus spp., Bacteroides spp. and Prevotella spp. (Breen, J. ed., The Gynecologist and the Older Patient, pp. 304-305 (1988)). Being polymicrobial in nature, BV etiology remains unclear. However, it is certain that BV involves the presence of a thick vaginal multi-species biofilm, where G. vaginalis is the predominant species. The decrease in the number of lactobacilli in the vagina has a dual effect, i.e., (i) a decreased competition for nutrients, and (ii) a decrease in the amount of lactic acid present, thus allowing for the multiplication of opportunistic pathogens in the vagina, whose growth is normally suppressed by the lactobacilli. Similar to what happens in many other biofilm-related infections, standard antibiotics, like metronidazole and clindamycin, are unable to fully eradicate the vaginal biofilm, which can explain the high recurrence rates of BV (Microbiol. 2016; 6:1528. Bacterial Vaginosis Biofilms: Challenges to Current Therapies and Emerging Solutions). Furthermore, antibiotic therapy can also cause a negative impact on the healthy vaginal microflora. These issues sparked the interest in developing alternative therapeutic strategies

BV is considered a broad spectrum infection requiring a broad spectrum treatment. Recurrence of resistant BV-associated pathogens is seen in more than 50% of women up to a year following treatment with antibiotics. Reactive oxygen species (ROS) have been in use for topical or local application to wounds, mucosa or internal structures where there may be heavy bacterial bioburden with biofilm and chronic inflammation. ROS has been successful in treating chronic wounds and in clearing multidrug-resistant organisms. One of the examples of ROS is Benzoyl peroxide (BPO). BPO is an organic peroxide included on the World Health Organization (WHO) Model Lists of Essential Medicines. A BPO-encapsulated hydrogel formulation has been shown capable of inhibiting the growth of the BV-associated pathogen G. vaginalis while having a limited effect on healthy lactobacilli in the vaginal ecosystem (Infect Dis Obstet Gynecol., Benzoyl Peroxide Formulated Polycarbophil/Carbopol 934P Hydrogel with Selective Antimicrobial Activity, Potentially Beneficial for Treatment and Prevention of Bacterial Vaginosis. v2013, Article ID 909354, 10 pages, 2013.)

The present invention discloses a method of treating or preventing a pathogenic bacterial infection by utilizing ROS in a concentration sufficient to avoid killing of target pathogen bacterial or triggering biofilm formation while arresting pathogenesis.

### SUMMARY

The description provides methods and compositions for treating or preventing bacterial infections and other disease and conditions caused by bacterial imbalance or mediated via human microbiota. The methods and compositions relate to the observation that sub-MIC of ROS, although too low to kill the target pathogenic bacteria, can interfere with quorum sensing to prevent pathogens from going into their virulent state. By preventing pathogens from disease-state virulence, biofilm formation is prevented, a proper balance of bacteria on a biological tissue surface (e.g., mucosal surface) is restored and maintained, the disease symptoms are treated and the disease recurrence is avoided.

Thus, in certain aspects, the description provides a method of treating or preventing a bacterial infection or modifying microbiota of a biological tissue surface comprising administering to a subject in need thereof a composition comprising a therapeutically effective amount of an ROS, wherein the therapeutically effective amount of the ROS is sufficient to avoid killing of disease-associated pathogens or triggering biofilm formation induced by oxidative stress in the bacteria while arresting disease pathogenesis.

In an additional aspect, the description provides a method of treating a microbial infection or modifying microbiota of a biological tissue surface in a subject comprising administering to a subject in need thereof a composition comprising a sub-minimal inhibitory concentration (MIC) of a reactive oxygen species (ROS), wherein the sub-MIC is sufficient to avoid killing or inhibiting microbial growth or triggering oxidative stress in the microbes but sufficient to arrest or reduce pathogenesis, and wherein the method effectuates the treatment of the microbial infection or a benefical modification of microbiota.

In any of the aspects or embodiments described herein, the biological tissue surface is at least one of gastrointestinal, lung, vaginal, skin, wound or combination thereof.

In certain embodiments, the pathogenic bacterial infection is characterized by an imbalance in the population of pathogenic bacterial as compared to probiotic bacteria.

In any of the aspects or embodiments described herein, the effective amount of the ROS is a sub-MIC of the ROS.

In any of the aspects or embodiments described herein, the sub-MIC of the ROS is sufficient for arresting virulence through inhibition of bacterial QS.

In any of the aspects or embodiments described herein, the inhibition of QS effectuates restoration of microbiota to non-pathogenic status.

In any of the aspects or embodiments described herein, the composition is a tablet, coated tablet, gel, cream, insert, vaginal ring or pessary. In certain embodiments, the composition is formulated for topical administration.

In any of the aspects or embodiments described herein, the ROS include oxygen ion free radicals (e.g. superoxide and hydroxyl radicals), and peroxides (e.g. hydrogen peroxide, benzoyl peroxide).

In certain embodiments, the ROS is benzyl peroxide (BPO). In certain embodiments, the composition as described herein comprises a hydrogel containing BPO

In any of the aspects or embodiments described herein, the composition does not decrease pH of a biological tissue surface or bacterial environment.

In any of the aspects or embodiments described herein, the biological tissue surface is at least one of gastrointestinal, lung, oral, vaginal, skin or combination thereof.

In any of the aspects or embodiments described herein, the bacterial infection is at least one of bacterial vaginosis, atopic dermatitis, cystic fibrosis or irritable bowel disease.

In any of the aspects of embodiments decrcribed herein, the conditions impacted by modification of microbiota are contributing to rheumatoid diseases such as rheumatoid arthritis, chronic skin conditions such as psoriasis, autoimmune diseases, systemic inflammation, cognitive impairment, and cardiovascular events.

In a certain embodiments, the bacterial infection is bacterial vaginosis.

In any of the aspects or embodiments described herein, the composition is co-administered with at least one other antibiotic treatment.

In another aspect, the description provides a pharmaceutical composition for use in treating or preventing a bacterial infection comprising an effective amount of a sub-MIC of an ROS, wherein the amount is sufficient to avoid killing or triggering oxidative stress while arresting pathogenesis through inhibition of QS.

In certain embodiments, the pharmaceutical composition is formulated for topical administration to a biological tissue surface.

In certain embodiments, the pharmaceutical composition is formulated for controlled release of a sub-MIC of a ROS.

In any of the aspects or embodiments described herein, the pharmaceutical composition comprises an effective amount of a sub-MIC of an ROS. In certain embodiments, the effective amount of BPO is between 125 μg/mL to 250 μg/mL.

These and other features of the systems and methods of the subject disclosure will become more readily apparent to those skilled in the art from the following detailed description taken in conjunction with the drawings.

### BRIEF DESCRIPTION OF THE DRAWINGS

FIG. 1 represents a graph disclosing different BPO concentrations in which biofilm production was inhibited.

### DETAILED DESCRIPTION

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

Although any methods and materials similar or equivalent to those described herein can also be used in the practice or testing of the present invention, the preferred methods and materials are now described. All publications mentioned herein are incorporated herein by reference to disclose and described the methods and/or materials in connection with which the publications are cited.

Unless otherwise defined, all technical and scientific terms used herein have the same meaning as commonly understood by one of ordinary skill in the art to which this invention belongs. The following references, the entire disclosures of which are incorporated herein by reference, provide one of skill with a general definition of many of the terms (unless defined otherwise herein) used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, the Harper Collins Dictionary of Biology (1991). Generally, the procedures of molecular biology methods described or inherent herein and the like are common methods used in the art. Such standard techniques can be found in reference manuals such as for example Sambrook et al., (2000, Molecular Cloning—A Laboratory Manual, Third Edition, Cold Spring Harbor Laboratories); and Ausubel et al., (1994, Current Protocols in Molecular Biology, John Wiley & Sons, New-York).

The terminology used in the description is for describing particular embodiments only and is not intended to be limiting of the invention. Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

The following terms are used to describe the present invention. In instances where a term is not specifically defined herein, that term is given an art-recognized meaning by those of ordinary skill applying that term in context to its use in describing the present invention.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The terms “co-administration”, “co-administered” and “co-administering” or “combination therapy” refer to both concurrent administration (administration of two or more agents at the same time) and time varied administration (administration of one or more agents at a time different from that of the administration of an additional agent or agents), as long as the agents are present in the area to be treated to some extent, preferably at effective amounts, at the same time. In certain preferred aspects, one or more of the present agents described herein, are co-administered in combination with at least one additional bioactive agent, especially including an antifungal, antibacterial, and/or biocide. In particularly preferred aspects, the co-administration of agents

The term “Reactive oxygen species” or “ROS”, as understood and used herein, refers to highly reactive and toxic oxygen compounds that are generated in the course of normal electron transport system during respiration or that are generated in a disease or during treatment with certain therapeutic agents for a particular disorder. ROS include, but are not limited to, the superoxide anion (O₂⁻), hydrogen peroxide (H₂O₂), benzoyl peroxide (BPO), singlet oxygen, lipid peroxides, and peroxynitrite

The term “Therapeutically effective amount” means the amount required to achieve a therapeutic effect. The therapeutic effect could be any therapeutic effect ranging from prevention, symptom amelioration, symptom treatment, to disease termination or cure.

The term “minimum inhibitory concentration” or “MIC”, as used herein, refers to the lowest concentration of an antimicrobial drug that will inhibit the visible growth of a microorganism. The MIC of a chemical is determined by preparing solutions of the chemical at increasing concentrations, incubating the solutions with the separate batches of cultured bacteria, and measuring the results using agar dilution or broth microdilution, usually following the guidelines of a reference body.

The term “quorum sensing” or “QS” refers to regulation of gene expression in response to fluctuations in cell-population density. Quorum sensing bacteria produce and release chemical signal molecules called autoinducers that increase in concentration as a function of cell density. The detection of a minimal threshold stimulatory concentration of an autoinducer leads to an alteration in gene expression. Gram-positive and Gram-negative bacteria use quorum sensing communication circuits to regulate a diverse array of physiological activities. These processes include symbiosis, virulence, competence, conjugation, antibiotic production, motility, sporulation, and biofilm formation.

The term “microbiota” refers to an ecological community of commensal, symbiotic and pathogenic microorganisms found in and on all multicellular organisms studied to date from plants to animals. A microbiota includes bacteria, archaea, protists, fungi and viruses. It is a collective term for the micro-organisms that live in or on the human body. Specific clusters of microbiota are found on the skin or in the gastrointestinal tract, mouth, vagina and eyes.

The term “bacterial vaginosis” or “BV” is a type of vaginal inflammation caused by the overgrowth of bacteria naturally found in the vagina, which upsets the natural balance. Normally, there are a lot of “good” bacteria and some “bad” bacteria in the vagina. The good types help control the growth of the bad types. In women with bacterial vaginosis, the balance is upset. There are not enough good bacteria and too many bad bacteria.

The term “topical administration” refers to a means of application to body surfaces such as the skin or mucous membranes to treat ailments via a large range of classes including creams, foams, gels, lotions, and ointments. Many topical medications are epicutaneous, meaning that they are applied directly to the skin.

Reference will now be made in detail to exemplary embodiments of the invention. While the invention will be described in conjunction with the exemplary embodiments, it will be understood that it is not intended to limit the invention to those embodiments. To the contrary, it is intended to cover alternatives, modifications, and equivalents as may be included within the spirit and scope of the invention as defined by the appended claims.

Exemplary compositions and methods of the present invention are described in more detail below.

Current invention discloses a method of treating and preventing infections of biological surfaces (gut-, lung-, oral- and vaginal surfaces, skin, wound surface) characterized by bacterial imbalance, such as Bacterial Vaginosis, Atopic Dermatitis, Cystic Fibrosis, Irritable Bowel Disease, and conditions and complications associated with such diseases that are associated with Bacterial Vaginosis through modification of microbiota by application of sub-oxidative-stress concentrations of ROS, that are:

Below conventionally used concentration that would kill the target pathogenic bacteria

Low enough for not inducing oxidative stress in lead pathogenic bacteria, and therefore not triggering biofilm formation

Simultaneously high enough for interfering with Quorum Sensing and acting as signaling molecules, preventing pathogens from going into their virulent state and from biofilm formation

Conditions like infertility, Preterm Delivery, Premature Rupture of Membranes, increased risk of acquiring of HIV are associated with Bacterial Vaginosis.

The anti-infective agents are the biologically active small molecules, which can interact with their microbial targets on different functional levels, dependent on the concentration of the agent. In therapeutic practice, the anti-infectives are used overwhelmingly at or above the “kill” levels aimed at either suppressing microbial functions and growth or at quick extermination of the microbial community (these concentration are typically defined as MIC, Minimal Inhibitory Concentrations and MBC, Minimal Bactericidal Concentrations).

Although it is acknowledged that at low concentrations the antimicrobial agents can exert biological activities other than killing microbes (in an above MBC), or preventing the population growth (in and above MIC), they are not therapeutically used in concentration corresponding to concentrations below MIC. Studies have established that typically, 1 to 2 log higher MIC concentrations are used in a clinical formulation as compared to concentration established in vitro.

Both the conventional thinking and experimental experience suggests that such use would not be effective in eliminating the pathogens and might stimulate antimicrobial resistance, both unwanted effects.

Both the internal, coming from within the community of microorganisms and the external oxidative stress could be the trigger for microbial imbalance. It could be assumed that there should be a certain level of such a stress, that would be below the level of “killing” but above the stress level, which would modify the behavior of microbes consistent with the “under-stress” circumstances.

The so-called pathogens, or potentially harmful bacteria, are often the part of “normal” microbiota (the constellation of microbial communities commensal for certain type of biological surface). When certain pathogens come under oxidative stress of a certain level, they might try to escape the stress by forming the biofilm.

Reactive Oxygen Species (ROS) are chemically reactive by-products of several metabolic processes that are routinely generated by microorganisms in aerobic conditions, and have evolutionarily developed scavenger mechanisms to protect them from damaging effect of oxidation. When the balance between ROS and scavenger system is disturbed, or when the scavenger system is overwhelmed with the level of ROS, the intercellular conditions called oxidative stress could lead to the dearth of microorganisms.

Medicinal products containing ROS have long been used for treatment or prophylaxis of infections. Examples: hydrogen peroxide in treatment/prophylaxis of wound infections; benzoyl peroxide in treatment of acne), or proposed/patented for treatment/prevention (peroxides for treatment/prevention of Bacterial Vaginosis as in the U.S. Pat. No. 7,709,026).

The prescribed/recommended concentrations at which ROS and other antimicrobials are used would typically be above the “kill” concentrations (MBC), where oxidative stress inevitably leads to the death of target pathogens (but also of many microorganisms, including non-pathogenic commensals.). In theory, the concentrations used could be also above the MIC (level where oxidative stress would prevent the population growth) but, because the mechanism of antimicrobial action of ROS, although purely understood, is associated rather with killing of microorganisms than with the prevention of their growth, the MIC concentrations are not typically considered in the context of antimicrobial use of ROS.

Current hypothesis formulated for the bacterial imbalance-associated diseases of mucosa and skin, entails the following assumptions:

The drug formulations of ROS are intended to treat the disease through the oxidative stress (impact). The impact of oxidation is universal, regardless of whether such an oxidation comes from the drug or has natural causes.

The degree of this stress/impact is related to the concentration of ROS species in the formulation, and is always disease- and formulation-specific. However, regardless of the formulation, the oxidative stress could conceptually be of three different levels of severity:
1. Kill level (MBC and above)
2. Escape-to-biofilm-level (MIC and above).
3. Below MIC level.
4. Below the QS-inhibiting level.

Level 1: Kill Level, MBC and Above.

At this level the oxidation overwhelms the scavenger and other defenses of pathogens and eventually leads to their death. Both the current therapeutic use of ROS-drugs and intended use are disclosed in the U.S. Pat. No. 7,709,026 for this level.

Level 2: Escape-to-Biofilm, MIC and Above.

The range of concentration (below the kill level 1) at which pathogens' scavenger defenses will hold, at least perhaps partially. At this level microbial cell survive oxidation but their growth is inhibited. Importantly, the oxidative stress of this level will trigger/stimulate the biofilm formation. Such a biofilm protects pathogens from excessive oxidative stress. Such a biofilm would achieve the oxidative equilibrium at the expense of modified composition of microbial community (certain pathogens will overgrow the beneficial microbes and other pathogens), and will increase virulence of lead pathogens.

The escape by lead pathogens from the “stress” levels of oxidative stress is the core driver behind the biofilm formation and Quorum Sensing mediated activation of virulence factors, and therefore behind the disease initiation and its recurrent nature. In other words, pathogens “escape” into biofilms from oxidative stress, thus transforming this microbial community, or microbiota, into an imbalanced (unhealthy) state. Such a microbiota is no longer “normal”. It is now (1) functionally dominated by pathogens that (2) have been activated to the disease level of virulence. In patients suffering from the diseases associated with microbial imbalanced, this is correspondently reflected in (1) microbiological finding (microbial imbalance, overgrowth of lead pathogens and decrease of other bacteria) and (2) clinical finding (symptoms of disease, reflection of achieved level of virulence).

Reactive Oxygen Species can produce changes consistent with the oxidative stress as described above. The mechanism of oxidative stress could also be deployed as a pathway of a mechanism of action of other antibacterial agents, for instance antibiotics. The key difference between ROS and antibiotics is that microorganisms, and especially microorganisms that live in the anaerobic environment with different levels of oxygen availability (e.g. gut or vaginal microbiota) do have the natural scavenger systems that protect them from unwanted oxidative stress (in other words, ROS and ROS-based drugs are “natural” enemies of microbes, and microbes do have the defense systems against ROS-based drugs).

The currently used ROS-drugs are not intended to be used at this level. However, all antibacterial drugs, ROS including, would be used at this level inadvertently, when the concentrations of the drug fluctuate due to release kinetics, at the whole target biological purpose or in a certain areas of it (e.g., hard to reach areas, very sick biofilm areas). Correspondently, and paradoxically, the ROS-drug in level 1 and 2 concentrations would therefore not only eliminate the pathogens (acting at level 1) but would also trigger the biofilm formation (acting at level-2). This is why the use of such drugs at these two levels would be associated with frequent disease recurrence.

Level 3: Below the MIC, but Above the Level at which ROS Will Inhibit QS

At this level the oxidative stress is compensated by the scavenger defense systems and microbial growth is not inhibited. A specific threshold below which biofilm formation will not be triggered is both pathogen and disease specific.

ROS would be conventionally used to “kill” pathogens (at level 1) and this would allow eliminating most of pathogens in short term. However, because some pathogens would in fact be only exposed to level 2 oxygenation, the biofilm will survive and quickly regrow. For the same reason ROS would not be used in the level 2. Current invention would work paradoxically at a level 3. This level represents a unique band of concentrations in which an ROS-drug will unexpectedly interfere with QS thus preventing the development of the disease-state virulence, hence will provide a “pacifying” signal to microbial communities, paradoxically treating and preventing disease. Because at this level sub-MIC concentrations will not promote escape-to-biofilm behavior, the use of ROS will result in reduction or elimination of recurrences and sustainable cure.

When applied in the disease- and formulation specific concentration in the above explained unique concentration band in 3rd level of concentrations, above the 4^{th} level of concentrations (in which QS-inhibiting threshold will not be achieved), ROS will work as signaling molecules that will interfere with QS.

This action will desensitize microbial community to external and internal escape-to-biofilm oxidative levels and to further oxidative stresses thus preventing biofilm formation and maintenance.

Thus, in certain aspects, the description provides a method of treating or preventing a microbial infection of a biological tissue surface comprising administering to a subject in need thereof a composition comprising a therapeutically effective amount of an ROS, wherein the therapeutically effective amount of the ROS is sufficient to avoid killing of microbes and inhibiting microbial growth or while arresting pathogenesis.

In an additional aspect, the description provides a method of treating a microbial infection or modifying microbiota of a biological tissue surface in a subject comprising administering to a subject in need thereof a composition comprising a sub-minimal inhibitory concentration (MIC) of a reactive oxygen species (ROS), wherein the sub-MIC is sufficient to avoid killing or inhibiting microbial growth or triggering oxidative stress in the microbes but sufficient to arrest or reduce pathogenesis, and wherein the method effectuates the treatment of the microbial infection or a beneficial modification of microbiota.

In any of the aspects or embodiments described herein, the biological tissue surface is at least one of gastrointestinal, lung, oral, vaginal, skin, wound or combination thereof.

In certain embodiments, the pathogenic bacterial infection is characterized by an imbalance in the population of microbes as compared to healthy microbiota.

In any of the aspects or embodiments described herein, the effective amount of the ROS is a sub-minimum inhibitory concentration (MIC) of the ROS.

In any of the aspects or embodiments described herein, the specific band of sub-MIC of the ROS (level 3 of concentration) is sufficient to inhibit bacterial quorum sensing (QS).

In any of the aspects or embodiments described herein, the inhibition of quorum sensing (QS) effectuates restoration of microbiota to non-pathogenic status.

In any of the aspects or embodiments described herein, the composition is a tablet, coated tablet, gel, cream, insert, vaginal ring or pessary. In certain embodiments, the composition is formulated for topical administration.

In any of the aspects or embodiments described herein, the ROS is benzyl peroxide (BPO). In certain embodiments, the composition as described herein comprises a hydrogel containing BPO

In any of the aspects or embodiments described herein, the composition does not directly decrease pH of a biological tissue surface or bacterial environment.

In any of the aspects or embodiments described herein, the biological tissue surface is at least one of gastrointestinal, lung, oral, vaginal, skin, wound or combination thereof.

In any of the aspects or embodiments described herein, the bacterial infection is at least one of bacterial vaginosis, atopic dermatitis, cystic fibrosis or irritable bowel disease.

In a certain embodiments, the bacterial infection is bacterial vaginosis.

In any of the aspects or embodiments described herein, the composition is co-administered with at least one other antibiotic or antimicrobial treatment.

In another aspect, the description provides a pharmaceutical composition for use in treating or preventing a bacterial infection comprising an effective amount of a specific sub-MIC of an ROS, wherein the amount is sufficient to avoid killing microbes or inhibiting microbial growth while arresting pathogenesis through inhibition of QS.

In certain embodiments, the pharmaceutical composition is formulated for topical administration to a biological tissue surface.

In certain embodiments, the pharmaceutical composition is formulated for controlled release of a sub-MIC of a ROS.

It has to be understood that MIC, MBC or any other concentrations established in-vitro in the process of pre-clinical development of topical drugs are not applied directly to the medicinal formulation that will be tested or eventually used in patients. Real-life conditions at any biologic surface are much more aggressive that during in-vitro testing. In patients any topical drug will be subject to physical elimination, dilution in biological fluids (e.g., vaginal fluid), aggressive enzymatic degradation, other biochemical activities leading to a loss of efficacy of active pharmaceutical ingredient and loss of vehicle structure. Therefore, the concentrations of anti-infective that are actually utilized in a drug would typically exceed the MIC by 10, 50, or 100 fold. For instance, if the MIC concentration for a certain BPO-containing gel was found to be 0.01%, it would very likely that concentrations that would be tested in humans would be about 0.5%, about 1%, and about 1.5%.

As such, in certain embodiments, the therapeutically effective amount or sub-MIC amount of the ROS, e.g., BPO, is less than or equal to about 5 wt %, less than or equal to about 4 wt %, less than or equal to about 3 wt %, less than or equal to about 2 wt %, less than or equal to about 1.5 wt %, less than or equal to about 1.25 wt %, less than or equal to about 1.0 wt %, less than or equal to about 0.75 wt %, less than or equal to about 0.5 wt %, or less than or equal to about 0.25 wt %. The skilled artisan would appreciate that such ranges necessarily exclude an amount of 0 wt %.

In any of the aspects or embodiments described herein, the pharmaceutical composition comprises an effective amount of a sub-MIC of an ROS. In certain embodiments, the effective amount is between 0.25 wt % to below 1 wt %

In any of the aspects or embodiments described herein, the pharmaceutical composition comprises an effective amount of a sub-MIC of an ROS. In certain embodiments, the effective amount of the ROS, e.g., BPO, is between about 100 μg/mL to 350 about μg/mL, or between about 125 μg/mL to 250 about μg/mL, between about 125 μg/mL to 225 about μg/mL, between about 125 μg/mL to 220 about μg/mL, between about 125 μg/mL to 215 about μg/mL, between about 125 μg/mL to 210 about μg/mL, between about 125 μg/mL to 200 about μg/mL, between about 125 μg/mL to 195 about μg/mL, between about 125 μg/mL to 190 about μg/mL, between about 125 μg/mL to 185 about μg/mL, between about 125 μg/mL to 180 about μg/mL, between about 125 μg/mL to 175 about μg/mL, between about 125 μg/mL to 170 about μg/mL, between about 125 μg/mL to 165 about μg/mL, between about 125 μg/mL to 160 about μg/mL, between about 125 μg/mL to 155 about μg/mL, between about 125 μg/mL to 150 about μg/mL, between about 125 μg/mL to 145 about μg/mL, between about 125 μg/mL to 140 about μg/mL, between about 125 μg/mL to 135 about μg/mL, between about 125 μg/mL to 130 about μg/mL, or about 125 μg/mL, or about 215 about μg/mL.

Application of ROC in such functionally-defined concentrations as level 3 is defined, will unexpectedly and paradoxically prevent the microbiota from internal and external oxidative stress (for instance associated with antimicrobial treatment or any other source of excessive oxygenation), and consequently from biofilm formation, hence breaking the vicious circle of disease recurrence.

This method of modification of microbiota without creating oxidative stress will lead to a sustainable cure and prevention of diseases associated with microbial imbalance.

When applied during the disease state, the medicinal application formulated based on this approach will treat the disease associated with microbiota imbalance by modifying the microbiota from the disease-state to “normal” (more commensal) without killing the microbes, without triggering the escape-to-biofilm behavior and without recurrent biofilm and disease symptoms associated with other methods of treatment.

When applied during the disease state concomitantly with other available methods of treatment or prior to the other method of treatment, the medicinal application formulated based on this approach would help to prevent the microbiota imbalance associated with other methods of treatment, increasing the effectiveness of treatment and helping to reduce the recurrence of the disease symptoms.

When applied after the initial treatment phase (which was performed with medicinal treatment involving the same approach or with other available methods of treatment), or this approach will restore microbiota to the health state and prevent the microbiota from relapsing in the disease state helping to reduce the recurrence of the disease or preventing the disease.

In terms of formulation, this could be any formulation or device (tablet, covered tablet, gel, cream, insert, vaginal ring or pessary, or any devise build from the specially-designed material) that would deliver ROS to the target biological surface predominantly in disease- and formulation-specific purpose-designed band of concentrations that are above the level inhibiting the QS but below the conventionally used concentration that would “kill” pathogens or inhibit their growth. Low enough for not inducing oxidative stress on pathogenic bacteria that could trigger biofilm formation but simultaneously high enough for interfering with QS and acting as signaling molecules, preventing pathogens from going into their virulent state.

### Example 1

BPO, in low sub-MIC could sufficiently interfere with QS of G. vaginalis, controlling G. vaginalis and preventing this pathogenic bacterium from reaching its virulent state.

Experimental Details: At a first step of the experiment, a broth microdilution assay was used to determine MIC and sub-MICs of BPO against G. vaginalis, a leading microorganism responsible for Bacterial Vaginosis. A concentration of 250 μg/mL was established as sub-MIC for G. vaginalis (in other words, concentration greater than 250 μg/mL of BPO was required to completely inhibit the growth of G. vaginalis).

As a second step, the concentrations, which inhibited biofilm production, were determined. Several concentrations of BPO were tested: 0, 31.25, 62.5, 125, and 250 μg/mL. A colorimetric method was used for biofilm staining and to determine the biofilm integrity percent after the treatment.

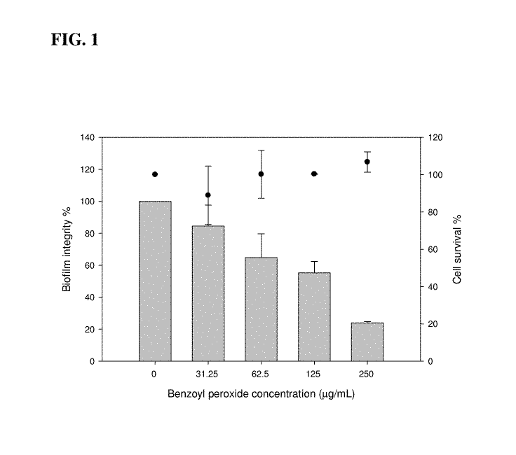

Results: Approximately 80% of biofilm formation was inhibited when the cells were treated with a sub-MIC concentration (250 μg/mL) of BPO compared with the control as shown in FIG. 1. There was more than 40% of biofilm prevention when a concentration of 125 μg/mL of BPO. Interestingly, the bacterial cells viability was not influenced by BPO, even when the highest sub-MIC concentration of 250 μg/mL was used. BPO in low sub-MICs would not trigger oxidative-stress related building or sustaining the biofilm (associated with development and recurrence of Bacterial Vaginosis). By using low, sub-MICs of BPO that would interfere with QS of G. vaginalis but would not create oxidative stress that would be strong enough to trigger biofilm formation, but instead would prevent biofilm formation, and hence would effectively treat and prevent Bacterial Vaginosis.

This data demonstrates that in a certain sub-MIC concentration BPO does not affect bacterial growth of G. vaginalis and partially inhibits G. vaginalis partial (fully consistent with sub-MIC definition) but, unexpectedly, very effectively inhibits the biofilm formation. This latter finding is against the established rationale of antibiotic treatment in which biofilm is expected to be more resistant to antibacterial treatment than the biofilm-forming microorganism and one is encouraged to treat biofilm with the higher dose not a lower dose.

With the reference to oxidative stress levels described in [0067], it can be assumed that:
1. Kill level (MBC and above)
2. Escape-to-biofilm-level (MIC and above): higher than 250 μg/mL.
3. Below MIC level: around 250 μg/mL (does not influence viability and only partically inhibits G. vaginalis but unexpectedly inhibits biofilm (20% of biofilm integrity).
4. Below the QS-inhibiting level: 125 μg/mL is likely to be out of the range or very close to be out, as the biofilm is inhibited but not as severely.

Note that in this test, the prototype of the vaginal gel was not used, but a DMSO solution of PBO (solution required for 96-well microplate) was used. Consequently, the inhibitory concentration for G. vaginalis found in this test cannot be compared with the inhibitory concentrations for BPO-gel as found in the previous experiments (Xu et al., 2013).

By using any ROS in a unique window of concentration that would interfere with QS but not trigger the biofilm formation (by lead pathogens) would effectively treat and prevent other disease and conditions associated with bacterial imbalance.

### Example 2. ROS in Level 3

The objective of finding a therapeutically effective concentration of an antibacterial drug (in fact, of any drug), cannot be achieved in pre-clinical studies (e.g. in-vitro or in-vivo), and is always a subject of clinical studies (studies in humans). In short, different concentrations would be tested in humans both for the treatment effect and for the safety (adverse effects), and the concentrations would be considered the best combination of efficacy and safety would be picked us therapeutic. The FDA wants drug companies to demonstrate both ends of the spectrum: low/insufficient efficacy on the low end, and signs of increased toxicity/compromised safety on high end. In present case, an efficacy in the low end of the spectrum, which is paradoxical and very unusual has been determined. In practice, that would mean that, for instance, a certain BPO-containing gel in 0.5% concentration would work better for treatment or prevention of Bacterial Vaginosis than the same gel in 0.1%, 1.0%, and 2.5% concentrations. For a different formulation, the sequence of concentrations could be different. For instance for a BPO-containing vaginal form we might be demonstrating that its 0.1% works better than its 0.05% and 0.5% concentrations. Note that study endpoints for establishing of efficacy would certainly be very different from that in in-vitro testing. For instances, such endpoints could be the proportion of patients with recurrence of Bacterial Vaginosis after certain duration of preventive treatment (in case of prevention goal of the study), or proportion of the patients that demonstrated either improvement of symptoms and signs of the disease, in case of treatment goal of the study.

### Exemplary Embodiments

In certain aspects, the description provides a method of treating a microbial infection or modifying microbiota of a biological tissue surface in a subject or use of a composition comprising a sub-minimal inhibitory concentration (MIC) of a reactive oxygen species (ROS) for treating a microbial infection or modifying microbiota of a biological tissue surface in a subject, the method comprising administering to a subject in need thereof a composition comprising a sub-minimal inhibitory concentration (MIC) of a reactive oxygen species (ROS), wherein the sub-MIC is sufficient to avoid killing or inhibiting microbial growth or triggering oxidative stress in the microbes but sufficient to arrest or reduce pathogenesis, and wherein the method effectuates the treatment of the microbial infection or a benefical modification of microbiota.

In certain additional aspects, the description provides a method of treating a microbial infection of a vagina or use of a composition comprising a sub-minimal inhibitory concentration (MIC) of a reactive oxygen species (ROS) for treating a microbial infection in a vagina, comprising vaginally administering to a subject in need thereof a composition comprising a sub-minimal inhibitory concentration (MIC) of benzoyl peroxide (BPO) sufficient to avoid killing or inhibiting microbial growth or triggering oxidative stress while arresting pathogenesis thereby attenuating or interfering with bacterial quorum sensing (QS), and wherein the method effectuates the treatment of the microbial infection.

In certain additional aspects, the description provides a pharmaceutical composition for treating or preventing a bacterial infection comprising a topical formulation including a sub-minimal inhibitory concentration (MIC) concentration of reactive oxygen species (ROS) sufficient to avoid killing or inhibiting growth or triggering oxidative stress while arresting pathogenesis through inhibition of quorum sensing (QS); and a pharmaceutically acceptable carrier.

In any of the aspects or embodiments described herein, the microbial infection is characterized by an imbalance in the population of pathogenic microbes as compared to normal or commensal microbes.

In any of the aspects or embodiments described herein, the modification of micobiota effectuates the treatment of at least one of a systemic inflammatory disorder, rheumatoid arthritis, psoriasis, irritable bowel disease or a combination thereof.

In any of the aspects or embodiments described herein, the microbial infection is characterized by the presence of a biofilm or biofilm-forming microbes.

In any of the aspects or embodiments described herein, the sub-MIC of the ROS is sufficient to inhibit bacterial quorum sensing (QS) without inducing oxidative burst.

In any of the aspects or embodiments described herein, the sub-MIC of the ROS is sufficient to inhibit biofilm formation thereby arresting pathogenesis or development of the disease, or recurrence.

In any of the aspects or embodiments described herein, the inhibition of QS effectuates restoration of microbiota to non-pathogenic status.

In any of the aspects or embodiments described herein, the composition is a tablet, coated tablet, gel, cream, insert, vaginal ring or pessary.

In any of the aspects or embodiments described herein, the composition is formulated for topical administration.

In any of the aspects or embodiments described herein, the ROS is benzyl peroxide (BPO).

In any of the aspects or embodiments described herein, the composition comprises a hydrogel containing BPO.

In any of the aspects or embodiments described herein, the composition does not directly decrease pH of a biological tissue surface or bacterial environment.

In any of the aspects or embodiments described herein, the biological tissue surface is at least one of gastrointestinal, lung, oral, vaginal, skin, wound or combination thereof.

In any of the aspects or embodiments described herein, the bacterial infection is at least one of bacterial vaginosis, atopic dermatitis, cystic fibrosis-related or irritable bowel disease.

In any of the aspects or embodiments described herein, the composition is co-administered with at least one other antimicrobial treatment.

In any of the aspects or embodiments described herein, the composition is cream or gel formulated for topical administration to a biological tissue surface.

In any of the aspects or embodiments described herein, the composition is formulated for controlled release of a sub-MIC of a ROS.

In any of the aspects or embodiments described herein, the effective amount or sub-MIC amount is between 125 μg/mL and 250 μg/mL.

In any of the aspects or embodiments described herein, the effective amount is sufficient to treat at least one of a systemic inflammatory disorder, rheumatoid arthritis, psoriasis, irritable bowel disease or a combination thereof.

Those skilled in the art will recognize, or be able to ascertain using no more than routine experimentation, many equivalents to the specific embodiments of the invention described herein. Such equivalents are intended to be encompassed by the following claims. It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present invention will be apparent from the appended claims. Moreover, those skilled in the art will recognize, or be able to ascertain using no more than routine experimentation, many equivalents to the specific embodiments of the invention described herein. Such equivalents are intended to be encompassed by the following claims.

1. A method of treating a microbial infection or modifying microbiota of a biological tissue surface in a subject comprising administering to a subject in need thereof a composition comprising a sub-minimal inhibitory concentration (MIC) of a reactive oxygen species (ROS), wherein the sub-MIC is sufficient to avoid killing or inhibiting microbial growth or triggering oxidative stress in the microbes but sufficient to arrest or reduce pathogenesis, and wherein the method effectuates the treatment of the microbial infection or a beneficial modification of microbiota.

2. The method of claim 1, wherein the microbial infection is characterized by an imbalance in the population of pathogenic microbes as compared to normal or commensal microbes.

3. The method of claim 1, wherein the modification of micobiota effectuates the treatment of at least one of a systemic inflammatory disorder, rheumatoid arthritis, psoriasis, irritable bowel disease or a combination thereof.

4. The method of claim 1, wherein the microbial infection is characterized by the presence of a biofilm or biofilm-forming microbes.

5. The method of claim 1, wherein the sub-MIC of the ROS is sufficient to inhibit bacterial quorum sensing (QS) without inducing oxidative burst.

6. The method of claim 5, wherein the sub-MIC of the ROS is sufficient to inhibit biofilm formation thereby arresting pathogenesis or development of the disease, or recurrence.

7. The method of claim 5, wherein the inhibition of QS effectuates restoration of microbiota to non-pathogenic status.

8. The method of claim 1, wherein composition is a tablet, coated tablet, gel, cream, insert, vaginal ring or pessary.

9. The method of claim 8, wherein the composition is formulated for topical administration.

10. The method of claim 1, wherein ROS is benzyl peroxide (BPO).

11. The method of claim 8, wherein composition comprises a hydrogel containing BPO

12. The method of claim 1, wherein the composition does not directly decrease pH of a biological tissue surface or bacterial environment.

13. The method of claim 12, wherein biological tissue surface is at least one of gastrointestinal, lung, oral, vaginal, skin, wound or combination thereof.

14. The method of claim 1, wherein the bacterial infection is at least one of bacterial vaginosis, atopic dermatitis, cystic fibrosis-related or irritable bowel disease.

15. The method of claim 1, wherein the composition is co-administered with at least one other antimicrobial treatment.

16. A method of treating a microbial infection of a vagina comprising vaginally administering to a subject in need thereof a composition comprising a sub-minimal inhibitory concentration (MIC) of benzoyl peroxide (BPO) sufficient to avoid killing or inhibiting microbial growth or triggering oxidative stress while arresting pathogenesis thereby attenuating or interfering with bacterial quorum sensing (QS), and wherein the method effectuates the treatment of the microbial infection.

17. The method of claim 16, wherein the bacterial infection is at least one of bacterial vaginosis, atopic dermatitis, psoriasis, cystic fibrosis-related or irritable bowel disease.

18. The method of claim 16, wherein composition comprises a hydrogel containing BPO.

19. A pharmaceutical composition for treating or preventing a bacterial infection comprising
a topical formulation including a sub-minimal inhibitory concentration (MIC) concentration of reactive oxygen species (ROS) sufficient to avoid killing or inhibiting growth or triggering oxidative stress while arresting pathogenesis through inhibition of quorum sensing (QS); and
a pharmaceutically acceptable carrier.

20. The pharmaceutical composition of claim 19, wherein the composition is cream or gel formulated for topical administration to a biological tissue surface.

21. The pharmaceutical composition of claim 20, wherein the composition is formulated for controlled release of a sub-MIC of a ROS.

22. The pharmaceutical composition of claim 19, wherein ROS is Benzoyl peroxide.

23. The pharmaceutical composition of claim 19, wherein the effective amount is between 125 μg/mL and 250 μg/mL.

24. The pharmaceutical composition of claim 23, wherein the effective amount is sufficient to treat at least one of a systemic inflammatory disorder, rheumatoid arthritis, psoriasis, irritable bowel disease or a combination thereof.
